(19) United States
(12) Patent Application Publication
(10) Pub. No.: US 2022/0400911 A1
(43) Pub. Date: Dec. 22, 2022

(54) Portable Hand Dryer

(71) Applicant: Pa Samba Ceesay, Wake Forest, NC (US)

(72) Inventor: Pa Samba Ceesay, Wake Forest, NC (US)

(21) Appl. No.: 17/841,789

(22) Filed: Jun. 16, 2022

Related U.S. Application Data
(60) Provisional application No. 63/212,859, filed on Jun. 21, 2021.

(51) Int. Cl. A47K 10/48; F26B 9/00

(52) U.S. Cl. CPC A47K 10/48; F26B 9/003

(57) ABSTRACT

A portable hand dryer may be compact in design and include and rechargeable battery such that the portable hand dryer is easy to carry in a purse, pocket, or bag and may be used to dry hands quickly and with ease. The portable hand dryer may include a rechargeable battery in operable communication with a charging port, at least one fan in operable communication a DC motor, propeller or impeller, and a heating element. The housing may define inlets and outlets constructed and arranged to allow the portable hand dryer to draw air in and output heated air.

### CROSS-REFERENCE TO RELATED APPLICATIONS

This application claims the benefit of U.S. Provisional Application No. 63/212,759 filed Jun. 21, 2021.

### TECHNICAL FIELD

The embodiments generally relate to the field of portable heaters and fans.

### BACKGROUND

Commercial hand dryers found in public places such as public restrooms and kitchens are commonly stationary, bulky, loud, and heavy. Alternatively, paper towel is commonly used in similar places to allow patrons to dry their hands. Reducing reliance on paper towels and commercial hand dryers may be beneficial for the environment, and a portable hand dryer may improve ease of use and convenience for a user.

### SUMMARY

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

A portable hand dryer may be compact in design and include and rechargeable battery or external power source such that the portable hand dryer is easy to carry in a purse, pocket, or bag and may be used to dry hands quickly and with ease. The portable hand dryer may include a rechargeable battery in operable communication with a charging port, at least one fan in operable communication with a DC motor in operable communication with the rechargeable battery or external power source, a printed circuit board (PCB), and a housing defining inlets and outlets constructed and arranged to allow the portable hand dryer to draw air in and output heated air. The portable hand dryer may also include a heating element to heat incoming air and discharge warmed air. The portable hand dryer may include motion or proximity sensors constructed and arranged to turn the dryer on upon determination of the presence of a user's hands nearby the dryer.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates one variation of a portable hand dryer according to some embodiments described herein;

FIG. 2 illustrates a partially transparent view of one variation of a portable hand dryer according to some embodiments described herein;

FIG. 3 illustrates a partially transparent view of one variation of a portable hand dryer according to some embodiments described herein;

FIG. 4 illustrates a partially transparent view of one variation of a portable hand dryer according to some embodiments described herein; and

FIG. 5 illustrates a partially transparent view of one variation of a portable hand dryer according to some embodiments described herein.

### DETAILED DESCRIPTION

The specific details of the single embodiment or variety of embodiments described herein are to the described system and methods of use. Any specific details of the embodiments are used for demonstration purposes only and no unnecessary limitations or inferences are to be understood from there.

It is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

A portable hand dryer may be compact in design and include a rechargeable battery or external power source such that the portable hand dryer is easy to carry in a purse, pocket, or bag and may be used to dry hands quickly and with ease. The portable hand dryer may include a hollow housing enclosing several components to be described. The housing may be generally cylindrical in shape, but may include a variety of different forms, sizes, and arrangements. A first end cap and second end cap may complete the housing and close off the hollow of the housing. The housing may define a hollow or cavity in which may be disposed a rechargeable battery in operable communication with a charging port such that the battery may be charged. The charging port may be partially exposed outside of the housing such that a charging cord may plug into the device. The charging port may be USB or any other format suitable for charging the battery. A power button may be partially exposed outside of the housing and may allow a user to turn the device on or off. At least one fan may be in operable communication with a DC motor in operable communication with the rechargeable battery. A heating element or component may be disposed above or below a fan in operable communication with the DC motor such that, during operation, the portable hand dryer may draw in air, heat the air within the housing, and expel heated air through the outlets. A PCB may facilitate operable communication between the battery, DC motor, heating element, charging port, motion or proximity sensor, or power button. The housing may additionally define inlets and outlets in the wall of the housing, constructed and arranged to allow the portable hand dryer to draw air in and output heated air.

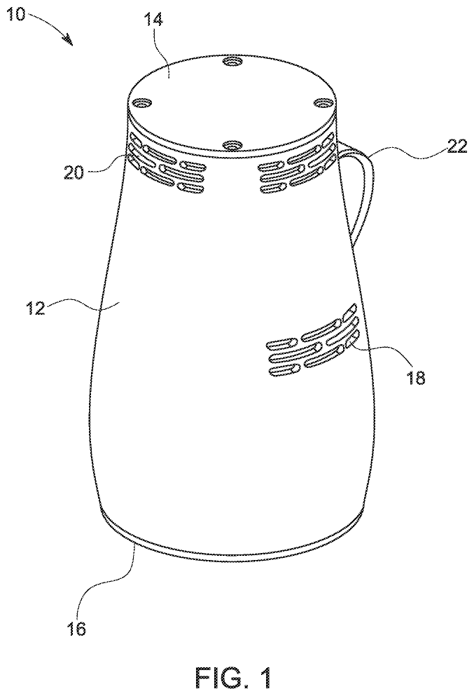

FIG. 1 depicts one variation of a portable hand dryer 10 including a hollow housing 12 defining a plurality of inlets 18 and a plurality of outlets 20 through which fluid such as air may flow. The portable hand dryer 10 may include at least one handle 22 which may allow a user to hold the portable hand dryer 10 or clip the portable hand dryer 10 to a belt, pocket, surface, or the like. A first end cap 14 and a second end cap 16 may close the housing 12.

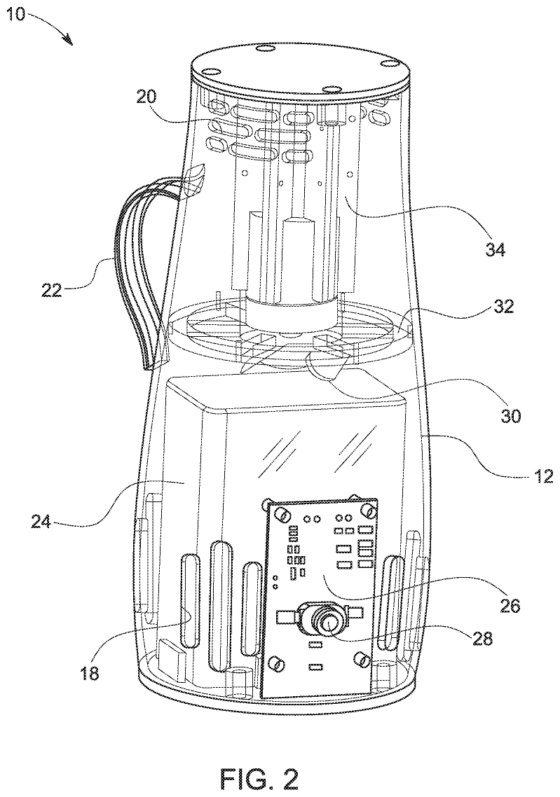

FIG. 2 depicts one variation of a portable hand dryer 10 illustrating a housing 12 for the purposes of depicting the internal components. The housing 12 is depicted as transparent for illustrative purposes. The portable hand dryer 10 including a hollow housing 12 defining a plurality of inlets 18 and a plurality of outlets 20 through which fluid such as air may flow. The portable hand dryer 10 may include a PCB 26 in operable communication with the various internal components described herein. A power button 28 may be in operable communication with the PCB 26 and may be constructed and arranged to allow a user to turn the portable hand dryer 10 on or off. A rechargeable battery 24 may be in operable communication with the PCB 26, a charging port, a DC motor 36, and a heating element 34. The DC motor 36 may be in operable communication with a fan propeller 30 or impeller constructed and arranged to, when on, draw air through the plurality of inlets 18, over the heating element 34, and out the plurality of outlets 20. A stabilizing bracket 32 may be constructed and arranged to secure the DC motor 36 to the housing 12 and reduce undesirably vibration of the DC motor 36 and propeller 30 during use.

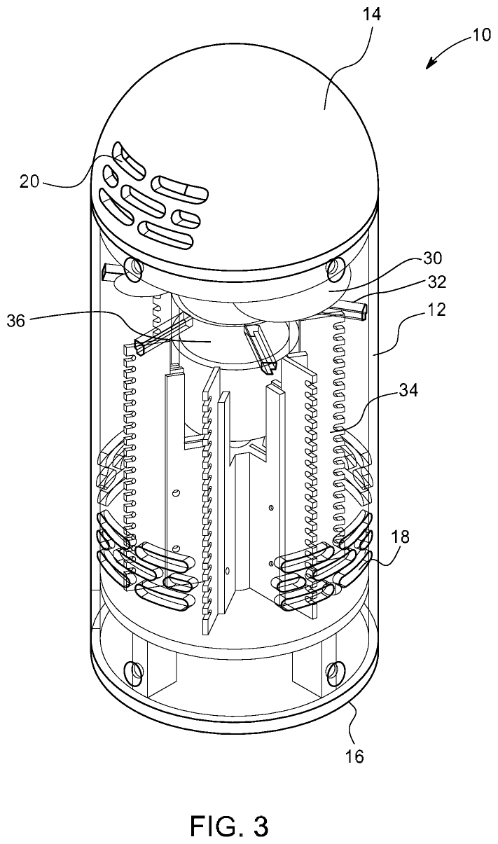

FIG. 3 depicts one variation of a portable hand dryer 10 illustrating a transparent housing 12 for the purposes of depicting the internal components. The housing 12 is depicted as transparent for illustrative purposes. The portable hand dryer 10 including a hollow housing 12 defining a plurality of inlets 18 and a plurality of outlets 20 through which fluid such as air may flow. A DC motor 36 may be in operable communication with a fan propeller 30 or impeller constructed and arranged to, when on, draw air through the plurality of inlets 18, over the heating element 34, and out the plurality of outlets 20. A stabilizing bracket 32 may be constructed and arranged to secure the DC motor 36 to the housing 12 and reduce undesirable vibration of the DC motor 36 and propeller 30 during use.

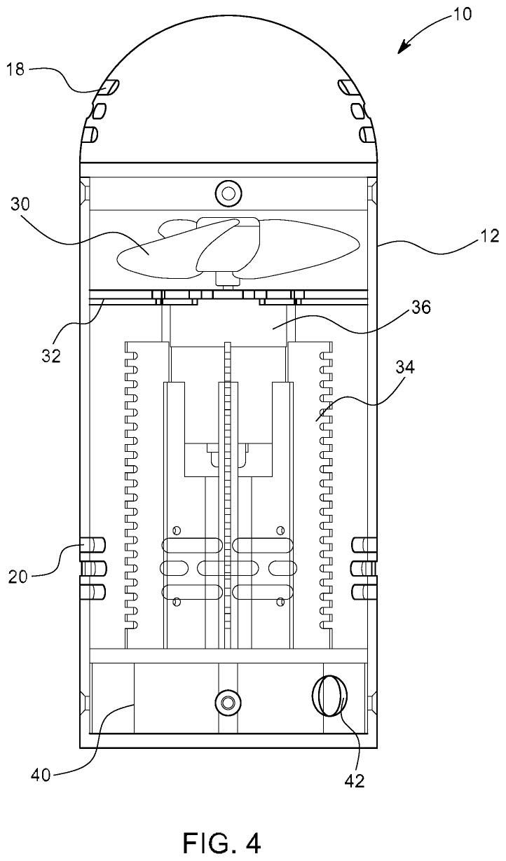

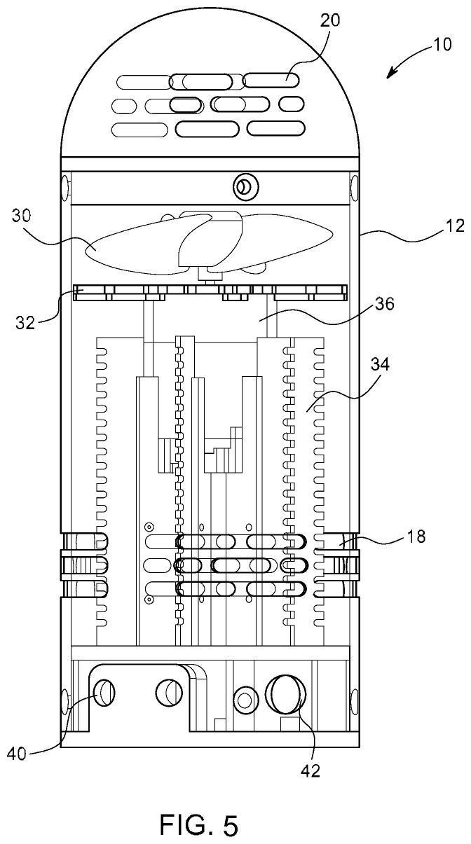

FIGS. 4 and 5 depict one variation of a portable hand dryer 10 illustrating a transparent housing 12 for the purposes of depicting the internal components. The housing 12 is depicted as transparent for illustrative purposes. The portable hand dryer 10 including a hollow housing 12 defining a plurality of inlets 18 and a plurality of outlets 20 through which fluid such as air may flow. A power button (not shown) may be in operable communication with a PCB (not shown) and may be constructed and arranged to allow a user to turn the portable hand dryer 10 on or off. A DC motor 36 may be in operable communication with a fan propeller 30 or impeller constructed and arranged to, when on, draw air through the plurality of inlets 18, over the heating element 34, and out the plurality of outlets 20. A stabilizing bracket 32 may be constructed and arranged to secure the DC motor 36 to the housing 12 and reduce undesirable vibration of the DC motor 36 and propeller 30 during use. At least one external power connector 42 may be constructed and arrange to operably connect with an external power source (not shown) to power the portable hand dryer 10. At least one motion or proximity sensor may be in operable communication with a PCB (not shown) and may be constructed and arranged to automatically turn on the portable hand dryer 10 when motion or an object with a predetermined proximity to the sensor is determined.

The following description of variants is only illustrative of components, elements, acts, products, and methods considered to be within the scope of the invention and are not in any way intended to limit such scope by what is specifically disclosed or not expressly set forth. The components, elements, acts, products, and methods as described herein may be combined and rearranged other than as expressly described herein and are still considered to be within the scope of the invention.

Variation 1 may include a portable hand dryer including a housing defining an internal cavity, a plurality of inlets, and a plurality of outlets; a first end cap and a second end cap at least partially enclosing the internal cavity; a PCB; a power button in operable communication with the PCB; a power source in operable communication with the PCB; a DC motor in operable communication with the PCB; a stabilizing bracket attached to the housing and being constructed and arranged to stabilize the DC motor; a heating element in operable communication with the PCB; and a fan propeller in operable communication with the DC motor, the fan propeller being constructed and arranged to draw a fluid through the plurality of inlets, over the heating element, and out the plurality of outlets.

Variation 2 may include a portable hand dryer as in variation 1, further including at least one handle affixed to the housing.

Variation 3 may include a portable hand dryer as in any of variations 1 through 2, further including at least one clip affixed to the housing.

Variation 4 may include a portable hand dryer as in any of variations 1 through 3, wherein the power source is an internal rechargeable battery.

Variation 5 may include a portable hand dryer as in any of variations 1 through 4, wherein the power source is an external power source.

Variation 6 may include a portable hand dryer as in any of variations 1 through 5, wherein the stabilizing bracket is constructed and arranged to reduce undesirably vibration of the DC motor and fan propeller during operation

Variation 7 may include a portable hand dryer as in any of variations 1 through 6, further including at least one external power connector constructed and arrange to operably connect with an external power source to power the portable hand dryer.

Variation 8 may include a portable hand dryer as in any of variations 1 through 7, further including at least one motion sensor in operable communication with a PCB, the at least one motion sensor being constructed and arranged to automatically turn on the portable hand dryer when motion of an object near the portable hand dryer is determined.

Variation 9 may include a portable hand dryer as in any of variations 1 through 8, further including at least one proximity sensor in operable communication with a PCB, the at least one proximity sensor being constructed and arranged to automatically turn on the portable hand dryer when an object within a predetermined proximity to the sensor is determined.

Variation 10 may include a portable hand dryer as in any of variations 1 through 9, wherein the housing is generally cylindrical.

Variation 11 may include a portable hand dryer as in any of variations 1 through 10, wherein the first cap is generally hemispherical.

Variation 12 may include a portable hand dryer including a generally cylindrical housing defining an internal cavity, a plurality of inlets, and a plurality of outlets; a hemispherical first end cap and a second end cap at least partially enclosing the internal cavity; a PCB; an internal rechargeable battery in operable communication with the PCB; a DC motor in operable communication with the PCB; a stabilizing bracket attached to the housing and being constructed and arranged to stabilize the DC motor; a heating element in operable communication with the PCB, the heating element being constructed and arranged to draw power from the internal battery to generate heat within the cavity; and a fan propeller in operable communication with the DC motor, the fan propeller being constructed and arranged to draw a fluid through the plurality of inlets, over the heating element, and out the plurality of outlets.

Variation 13 may include a portable hand dryer as in variation 12, further including at least one handle affixed to the housing.

Variation 14 may include a portable hand dryer as in any of variations 12 through 13, further including at least one clip affixed to the housing.

Variation 15 may include a portable hand dryer as in any of variations 12 through 14, wherein the stabilizing bracket is constructed and arranged to reduce undesirably vibration of the DC motor and fan propeller during operation.

Variation 16 may include a portable hand dryer as in any of variations 12 through 15, further including at least one motion sensor in operable communication with the PCB, the at least one motion sensor being constructed and arranged to automatically turn on the portable hand dryer when motion of an object near the portable hand dryer is determined.

Variation 17 may include a portable hand dryer as in any of variations 12 through 16, further including at least one proximity sensor in operable communication with the PCB, the at least one proximity sensor being constructed and arranged to automatically turn on the portable hand dryer when an object within a predetermined proximity to the sensor is determined.

Variation 18 may include portable hand dryer including a generally cylindrical housing defining an internal cavity, a plurality of inlets, and a plurality of outlets; a hemispherical first end cap and a second end cap at least partially enclosing the internal cavity; a PCB; at least one external power connector in operable communication with the PCB and being constructed and arrange to operably connect with an external power source to power the portable hand dryer; a DC motor in operable communication with the PCB; a stabilizing bracket attached to the housing and being constructed and arranged to stabilize the DC motor; a plurality of heating elements in operable communication with the PCB, the plurality of heating elements being constructed and arranged to draw power from the external power source to generate heat within the cavity; and a fan propeller in operable communication with the DC motor, the fan propeller being constructed and arranged to draw a fluid through the plurality of inlets, over the heating element, and out the plurality of outlets.

Variation 19 may include a portable hand dryer as in variation 18, further including at least one motion sensor in operable communication with the PCB, the at least one motion sensor being constructed and arranged to automatically turn on the portable hand dryer when motion of an object near the portable hand dryer is determined.

Variation 20 may include a portable hand dryer as in any of variations 18 through 19, further including at least one proximity sensor in operable communication with the PCB, the at least one proximity sensor being constructed and arranged to automatically turn on the portable hand dryer when an object within a predetermined proximity to the sensor is determined.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for anyone of the elements in the claims below or that a single element can be substituted for two or more elements in a claim. Although elements can be described above as acting in certain combinations, and even initially claimed as such, it is to be expressly understood that one or more elements from a claimed combination can, in some cases, be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible considering the above teachings without departing from the following claims.

What is claimed is:

1. A portable hand dryer comprising:
a housing defining an internal cavity, a plurality of inlets, and a plurality of outlets;
a first end cap and a second end cap at least partially enclosing the internal cavity;
a PCB;
a power button in operable communication with the PCB;
a power source in operable communication with the PCB;
a DC motor in operable communication with the PCB;
a stabilizing bracket attached to the housing and being constructed and arranged to stabilize the DC motor;
a heating element in operable communication with the PCB; and
a fan propeller in operable communication with the DC motor, the fan propeller being constructed and arranged to draw a fluid through the plurality of inlets, over the heating element, and out the plurality of outlets.

2. The portable hand dryer as in claim 1, further comprising at least one handle affixed to the housing.

3. The portable hand dryer as in claim 1, further comprising at least one clip affixed to the housing.

4. The portable hand dryer as in claim 1, wherein the power source is an internal rechargeable battery.

5. The portable hand dryer as in claim 1, wherein the power source is an external power source.

6. The portable hand dryer as in claim 1, wherein the stabilizing bracket is constructed and arranged to reduce undesirably vibration of the DC motor and fan propeller during operation.

7. The portable hand dryer as in claim 1, further comprising at least one external power connector constructed and arrange to operably connect with an external power source to power the portable hand dryer.

8. The portable hand dryer as in claim 1, further comprising at least one motion sensor in operable communication with a PCB, the at least one motion sensor being constructed and arranged to automatically turn on the portable hand dryer when motion of an object near the portable hand dryer is determined.

9. The portable hand dryer as in claim 1, further comprising at least one proximity sensor in operable communication with a PCB, the at least one proximity sensor being constructed and arranged to automatically turn on the portable hand dryer when an object within a predetermined proximity to the sensor is determined.

10. The portable hand dryer as in claim 1, wherein the housing is generally cylindrical.

11. The portable hand dryer as in claim 1, wherein the first cap is generally hemispherical.

12. A portable hand dryer comprising:
a generally cylindrical housing defining an internal cavity, a plurality of inlets, and a plurality of outlets;
a hemispherical first end cap and a second end cap at least partially enclosing the internal cavity;
a PCB;
an internal rechargeable battery in operable communication with the PCB;
a DC motor in operable communication with the PCB;
a stabilizing bracket attached to the housing and being constructed and arranged to stabilize the DC motor;
a heating element in operable communication with the PCB, the heating element being constructed and arranged to draw power from the internal battery to generate heat within the cavity; and
a fan propeller in operable communication with the DC motor, the fan propeller being constructed and arranged to draw a fluid through the plurality of inlets, over the heating element, and out the plurality of outlets.

13. The portable hand dryer as in claim 12, further comprising at least one handle affixed to the housing.

14. The portable hand dryer as in claim 12, further comprising at least one clip affixed to the housing.

15. The portable hand dryer as in claim 12, wherein the stabilizing bracket is constructed and arranged to reduce undesirably vibration of the DC motor and fan propeller during operation.

16. The portable hand dryer as in claim 12, further comprising at least one motion sensor in operable communication with the PCB, the at least one motion sensor being constructed and arranged to automatically turn on the portable hand dryer when motion of an object near the portable hand dryer is determined.

17. The portable hand dryer as in claim 12, further comprising at least one proximity sensor in operable communication with the PCB, the at least one proximity sensor being constructed and arranged to automatically turn on the portable hand dryer when an object within a predetermined proximity to the sensor is determined.

18. A portable hand dryer comprising:
a generally cylindrical housing defining an internal cavity, a plurality of inlets, and a plurality of outlets;
a hemispherical first end cap and a second end cap at least partially enclosing the internal cavity;
a PCB;
at least one external power connector in operable communication with the PCB and being constructed and arrange to operably connect with an external power source to power the portable hand dryer;
a DC motor in operable communication with the PCB;
a stabilizing bracket attached to the housing and being constructed and arranged to stabilize the DC motor;
a plurality of heating elements in operable communication with the PCB, the plurality of heating elements being constructed and arranged to draw power from the external power source to generate heat within the cavity; and
a fan propeller in operable communication with the DC motor, the fan propeller being constructed and arranged to draw a fluid through the plurality of inlets, over the heating element, and out the plurality of outlets.

19. The portable hand dryer as in claim 18, further comprising at least one motion sensor in operable communication with the PCB, the at least one motion sensor being constructed and arranged to automatically turn on the portable hand dryer when motion of an object near the portable hand dryer is determined.

20. The portable hand dryer as in claim 18, further comprising at least one proximity sensor in operable communication with the PCB, the at least one proximity sensor being constructed and arranged to automatically turn on the portable hand dryer when an object within a predetermined proximity to the sensor is determined.
